(19) United States
(12) Patent Application Publication
(10) Pub. No.: US 2004/0048840 A1
(43) Pub. Date: Mar. 11, 2004

(54) Modulation of matrix metalloproteinase (MMP) activity with aldosterone blocker(s)

(75) Inventors: Amy E. Rudolph, St. Louis, MO (US); Ellen G. McMahon, St. Louis, MO (US)

Correspondence Address: BANNER & WITCOFF, 1001 G STREET N W, SUITE 1100, WASHINGTON, DC, 20001, US

(73) Assignee: Pharmacia Corporation, St. Louis, MO (US)

(21) Appl. No.: 10/645,564

(22) Filed: Aug. 22, 2003

Related U.S. Application Data
(60) Provisional application No. 60/405,292, filed on Aug. 23, 2002.

(51) Int. Cl. A61K 31/585; A61K 31/56

(52) U.S. Cl. 514/177; 514/175

(57) ABSTRACT

The present invention is directed to a method for preventing an increase in matrix metalloproteinase (MMP) activity or reducing MMP activity in a subject in need thereof by administering to the subject a therapeutically effective amount of a selective aldosterone blocker. More particularly, the present invention is directed to attenuating or preventing an increase in MMP activity comprising administering eplerenone, or derivatives thereof.

This application is a non-provisional of provisional application Serial No. 60/405,292 filed Aug. 23, 2003, the contents of which is incorporated herein in its entirety.

### FIELD OF THE INVENTION

The present invention is directed to a method for preventing an increase in matrix metalloproteinase (MMP) activity or reducing MMP activity in a subject. More particularly, the present invention is directed to attenuating MMP activity or preventing an increase in MMP activity comprising administering eplerenone, or derivatives thereof, in a therapeutically effective amount to a subject in need thereof.

### DESCRIPTION OF THE RELATED ART

MMPs are a family of zinc-dependent endopeptidases that have the capacity to degrade the extracellular matrix (ECM). The controlled/coordinated breakdown of ECM permits normal operation of metabolic processes, proper operation of homeostatic degradation and repair of ECM to maintain structural and spatial tissue integrity and tissue morphology necessary to maintain normal life-sustaining functions.

A number of recent publications discuss matrix metalloproteinases (MMPs). See, for example, Terrence M. Doherty, et al., Therapeutic Developments in Matrix Metalloproteinase Inhibition, review article, Expert Opin. Cir. Patents, 12(5) pp. 665-707, Ashley Publications (2002).

While T. M. Doherty, et al., supra, recognize that MMPs play a role in a variety of pathologies such as tumor invasion, metastases, inflammatory diseases, arthritis, atherosclerosis, ventricular remodeling and cardiac rupture, to name a few, they indicate that the role of MMPs in modulating various body functions is a complex subject requiring further research and study.

Among the various pathologies in which MMPs play a role, cardiovascular diseases account for a large proportion of today's worldwide morbidity and mortality. For example, clinical studies have shown that the extent of left ventricular (LV) dilation in patients with heart failure (HF) is a strong predictor of morbidity and mortality. Further, increased MMP activity has been observed in the human failing myocardium and the myocardium in animal models of HF. Increased MMP activity can lead to accelerated degradation of the ECM which may facilitate LV dilation in HF progression. Accordingly, there is a need to provide a method for regulating MMP activity. More particularly, there is a need to provide a method to attenuate MMP activity or to prevent or reduce an increase in MMP activity.

### BRIEF DESCRIPTION OF THE DRAWINGS

FIG. 1a depicts a photograph of an electrophoresed gel depicting gelatinous activity in normal canines (n=7), placebo dosed canines (n=7) and eplerenone dosed canines (n=7).

FIG. 1b plots the gelatinase activity (quantitated using densitometry) exhibited in FIG. 1a in bar graph format.

### SUMMARY OF THE INVENTION

In view of the foregoing, the present invention provides a method for attenuating, reducing, or preventing an increase in MMP activity in a mammal, preferably a human. According to one embodiment of the invention, the present invention comprises a method for attenuating MMP activity comprising the step of:

(a) administering, to a subject (e.g., patient) in need thereof, a therapeutically effective amount of an epoxy steroidal compound.

Preferably, the epoxy steroidal compound is a selective aldosterone blocker such as eplerenone, or an isomeric or tautomeric form of a derivative thereof. The structure of eplerenone is represented by Formula (I) below:

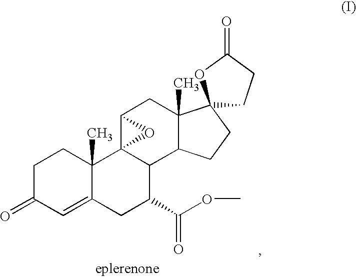

According to other embodiments of the present invention, a reduction or attenuation of MMP activity or prevention of an increase in MMP activity may be accomplished by administering one or more isomeric or tautomeric forms of derivatives of eplerenone, the derivatives represented by Formulas (II)-(X) below:

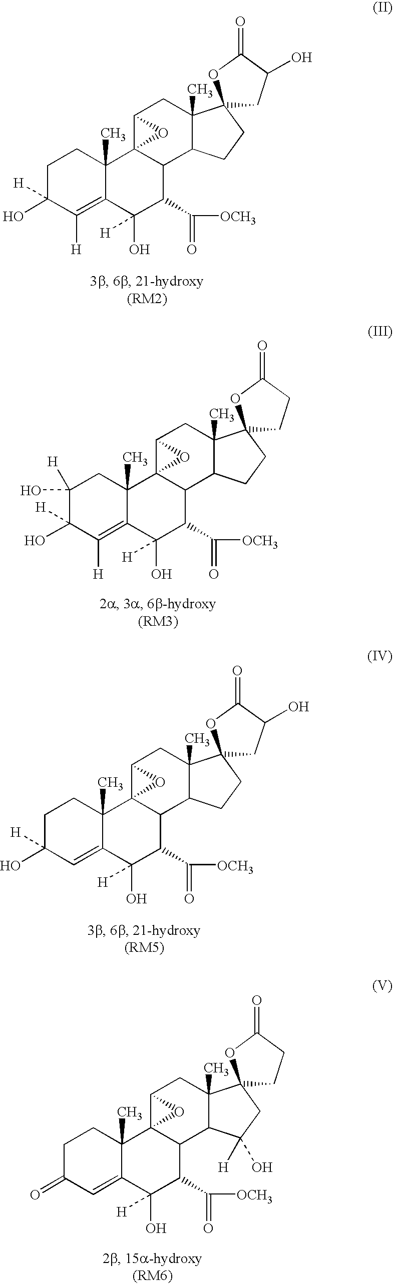

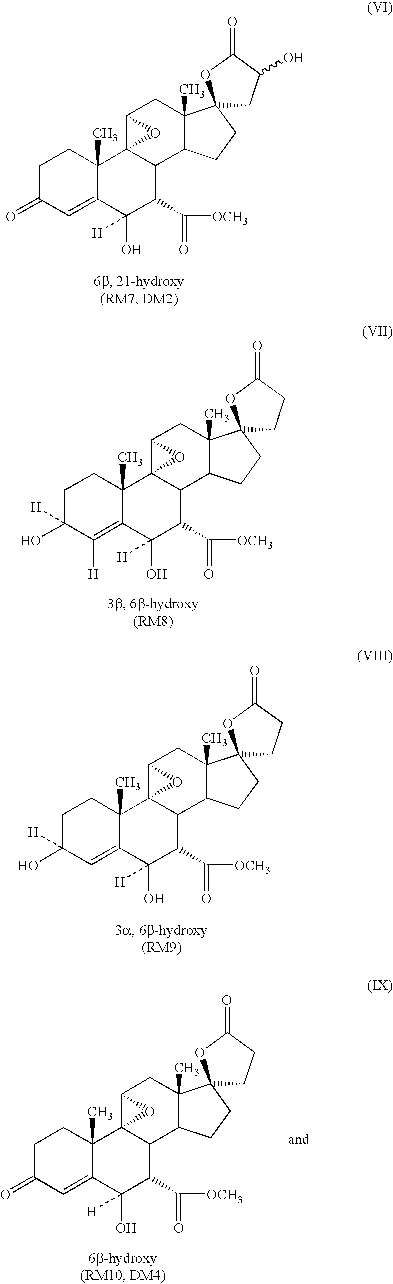

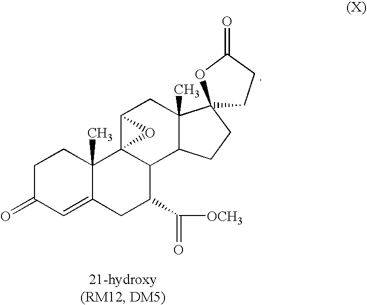

In further embodiments, also included in connection with use of the method(s) of the present invention are the isomeric forms and tautomers and the pharmaceutically-acceptable salts of the compounds of Formula (I) and/or isomeric or tautomeric forms of Formulas (II)-(X) and include their diastereomers, enantiomers, and racemates as well as their structural isomers.

### DETAILED DESCRIPTION OF THE PREFERRED EMBODIMENTS

The scope of the applicability of the present invention will become apparent from the detailed description presented below. However, it should be understood that the following detailed description and any examples, while indicating preferred embodiments of the invention, are provided for illustrative purposes only.

Further, the detailed description below is provided to aid those skilled in the art in practicing the present invention. This detailed description should not be construed to unduly limit the present invention as modifications and variations in the embodiments discussed herein can be made by those of ordinary skill in the art without departing from the spirit or scope of the present inventive discovery.

The term “aldosterone blocker” is synonymous with the term “aldosterone antagonist,” selective aldosterone antagonist,’ “selective receptor aldosterone antagonist,” “mineralocorticoid receptor antagonist,” mineralocorticoid receptor blocker,” and other synonyms thereof.

MMP Activity Modulation in Various Tissues

It has been discovered that eplerenone of Formula (I) and/or one or more of the isomeric or tautomeric forms of the derivative(s) of Formulas (II)-(X) are suitable for attenuating, reducing or preventing an increase in MMP activity. In particular, according to an embodiment of the present invention, administering a therapeutically effective amount of eplerenone reduces or prevents an increase in MMP activity of certain MMPs including, but not limited to, for example, MMP-2, MMP-9 and MMP-13. Preferably, MMP activity is modulated by the above-noted methods in myocardial tissue, left ventricular tissue, and/or in vascular tissue such as coronary vessels. Persons in need of such MMP activity modulation include those that exhibit signs and/or symptoms of acute renal failure, renal failure, renal disease (including end stage renal disease—ESDR), coronary artery disease, diabetes, syndrome X, stroke, heart failure including, but not limited to, hypertension, left ventricular hypertrophy, heart failure class-II, heart failure class-III, heart failure class-IV, cardiac fibrosis, atherosclerosis, enlargement of any portion of the heart, left ventricular dilation, progressive left ventricular failure, or having a reduced left ventricular ejection fraction (LVEF) less than about 35-40%. Preferably, the subject in need of treatment is a mammal and, more preferably, a human.

Therapeutically Effective Amount

As used herein, an “effective amount” or “therapeutically effective amount” means the dose or effective amount to be administered to a patient and the frequency of administration to the patient which is sufficient to obtain a reduction or attenuation of MMP activity or prevention of an increase in the MMP activity as readily determined by one of ordinary skill in the art, by the use of known techniques (e.g., Gelatin Zymography, Casein Zymography, etc.) used to measure MMP activity and by observing results obtained under analogous circumstances.

Daily Dosages

The dosage regimen for modulating MMP activity (with a compound and/or a composition of Formula (I) (and/or the isomeric or tautomeric forms of Formulas (II)-(X), respectively) in accordance with the present invention may be selected according to a variety of factors, including, but not limited to, the type, age, weight, sex, diet and medical condition of the patient, the severity of the disease, the route of administration, pharmacological considerations such as the potency, activity, efficacy, pharmacokinetic and toxicology profiles of the particular compound or composition employed, whether a drug delivery system is utilized and whether the compound is administered as part of a drug combination. Accordingly, the dosage regimen can vary widely and can deviate from the preferred dosage regimen set forth herein.

The eplerenone administered in accordance with an embodiment of the present invention may be provided in a single daily dose or in multiple divided sub-doses. The total daily dose administered (of the eplerenone (Formula (I)) or its derivatives (Formulas (II)-(X))) in single or in multiple divided sub-doses can be an amount, for example, from about 0.5 mg/kg/day to about 200 mg/kg/day based on a total body weight of a mammalian recipient (e.g., veterinary animals such as cats, dogs, mice, rats, etc.) and more usually from about 25 mg/day to about 400 mg/day in a human recipient. Those skilled in the art will appreciate that these dosages may also be adjusted with guidance from Goodman & Gillman's The Pharmacological Basis of Therapeutics, Ninth Edition (1996), and from Goodman & Gillman's The Pharmacological Basis of Therapeutics, Tenth Edition (2001).

Exemplary daily doses of eplerenone range from about 25 mg eplerenone to about 400 mg eplerenone provided in either a single daily dose or in multiple divided sub-doses. Other exemplary daily doses of eplerenone (on a mg/kg of recipient's body weight basis) range from about 3 mg/kg to about 300 mg/kg. Still more exemplary daily doses of eplerenone include, but are not limited to, 25 mg, 50 mg, 75 mg, 100 mg, 150 mg, 175 mg, 200 mg, 225 mg, 250 mg, 275 mg, 300 mg, 325 mg, 350 mg, 375 mg and 400 mg. Similar dosage ranges as those noted above (in conjunction with eplerenone) may be used in conjunction with the isomeric or tautomeric forms of the derivatives of Formulas (II)-(X), respectively. Multiple doses per day of the compound of Formula (I) (and/or the isomeric or tautomeric forms of Formulas (II)-(X), respectively) can also increase the total daily dose, should such doses be desired by the person prescribing the eplerenone compound.

The daily dosage of any of the compounds of Formula (I) (and/or the isomeric or tautomeric forms of Formulas (II)-(X), respectively), in accordance with the present invention should be a therapeutically effective amount sufficient to attenuate or reduce MMP activity or prevent an increase in MMP activity.

Pharmaceutical Compositions and Routes of Administration

Further, a compound useful in the present invention can be formulated as a pharmaceutical composition. Formulation of drugs and their adjuvants and excipients are provide in Remington's Pharmaceutical Sciences, Mack Publishing Company, Eastern, Pa. (1985) and its later editions. See, also, Lieberman, H. A. et al., eds. Pharmaceutical Dosage Forms, Marcel Decker, New York, N.Y. (1980).

Such pharmaceutical compositions can be administered orally, parenterally, by inhalation spray, rectally, or topically in dosage unit formulations containing conventional, non-toxic pharmaceutically acceptable additives, adjuvants and vehicles as desired.

Dosage Forms

The following Table lists various dosage forms of the pharmaceutical composition for use in conjunction with the method of the present invention.

TABLE

| No. | Exemplary Dosage Forms |
|---|---|
| | Oral dosage forms |
| 1. | Tablet |
| 2. | Slow Release Tablet |
| 3. | Effervescent Tablet |
| 4. | Enteric Coated Tablet |
| 5. | Compressed Tablet |
| 6. | Molded Tablet |
| 7. | Capsule |
| 8. | Slow Release Capsule |
| 9. | Capsule for Use in or with Nebulizer |
| 10. | Gelatin Capsule |
| 11. | Caplet |
| 12. | Troche |
| 13. | Powder |
| 14. | Lozenge |
| 15. | Gum |
| 16. | Solution |
| 17. | Suspension |
| 18. | Emulsion |
| 19. | Dispersion |
| | Parenteral Dosage Forms |
| 20. | Intramuscular Injection |
| 21. | Intravenous Injection |
| | Other Dosage Forms |
| 22. | Intra Cerebral Ventricular (ICV) Infusion |
| 23. | Osmotic Pump |
| 24. | Infusion Pump |
| 25. | Elixir |
| 26. | Injection |
| 27. | Pellets |
| 28. | Implants |
| 29. | Suppository |
| 30. | Syrup |
| 31. | Oral Gel |
| 32. | Oral Paste |

For a more complete list of dosage forms in addition to those provided in the Table above, see Remington's Pharmaceutical Sciences, Mack Publishing Co., Easton, Pa., Arthur Osol (editor), 16^{th} Edition (1980). Also see each of the later editions of the same (i.e., each later edition to date of Remington's Pharmaceutical Sciences). Also see, The United States Pharmacopeia, 21^{st} Edition, United States Pharmacopeial Convention, Washington, D.C. (1985). Also see each of the later editions of the same (i.e., each later edition to date of The United States Pharmacopeia).

Parenteral

In connection with the inventive method, the compound of Formula (I) (and/or the isomeric or tautomeric forms of Formulas (II)-(X), respectively) may be administered parenterally, either subcutaneously, or intravenously, or intramuscularly, or intrasternally, or by infusion techniques, in the form of sterile injectable aqueous or olagenous suspensions. Such suspensions may be formulated according to the known art using those suitable dispersing of wetting agents and suspending agents which have been mentioned above, or other acceptable agents. The sterile injectable preparation may also be a sterile injectable solution or suspension in a non-toxic parenterally-acceptable diluent or solvent. Among the acceptable vehicles and solvents that may be employed are water, Ringer's solution and isotonic sodium chloride solution.

In addition, sterile, fixed oils are conventionally employed as a solvent or suspending medium. For this purpose, any bland fixed oil may be employed including synthetic mono- or diglycerides. In addition, n-3 polyunsaturated fatty acids may find use in the preparation of injectables. The compounds can also be dissolved or suspended in polyethylene glycol, propylene glycol, ethanol, corn oil, cottonseed oil, peanut oil, sesame oil, benzoalcohol, sodium chloride and/or various buffers. Other adjuvants and modes of administration are well and widely known in the pharmaceutical art.

Oral

A pharmaceutical composition of one or more of the compounds of Formula (I) (and/or the isomeric or tautomeric forms of Formulas (II)-(X), respectively) in connection with the method(s) of the present invention can be administered orally, for example, as tablets, coated tablets, dragees, troches, lozenges, gums, aqueous or oily suspensions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, sucrose, calcium phosphate or sodium phosphate; granulating and disintegrating agents, for example, maize starch, or alginic acid; binding agents, for example, starch, gelatin or acacia gum; lubricating agents, for example magnesium stearate, stearic acid or talc; and buffering agents, for example, sodium citrate, magnesium or calcium carbonate or bicarbonate. The tablets may be uncoated or they may be coated by known techniques to delay disintegration and adsorption in the gastrointestinal tract and thereby provide a sustained action over a longer period. For example, a time delay material such as glyceryl monostearate or glyceryl distearate may be employed. Other exemplary excipients include cellulose esters of alkanoic acid, cellulose alkyl esters, magnesium oxide, sodium alginate, polyvinyl pyrrolidone, and/or polyvinyl alcohol, sodium and calcium salts of phosphoric acids and sulfuric acids.

Formulations for oral use may also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin, or olive oil. Preferably, when orally administered, the pharmaceutical composition may be at or near body temperature or ambient temperature.

Liquid dosage forms for oral administration can include, but not limited, pharmaceutically acceptable emulsions, solutions, suspensions, syrups and elixirs containing inert diluents commonly used in the art, for example, water. Such compositions can also comprise adjuvants, for example, wetting agents, emulsifying and suspending agents and sweetening, flavoring and perfuming agents. The amount of the active compound that can be combined with the carrier material comprise a single dosage form varies depending upon the mammalian host treated and the particular mode of administration.

Drug solutions, including aqueous and oil-based suspensions, can be produced that contain the active materials in admixture with excipients suitable for their manufacture. Such excipients are suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate.

The solutions, such as aqueous or oil-based suspensions may also contain one or more preservatives or stabilizers. In cases where the solution is used for oral administration, it may be desirable to use one or more coloring agents, one or more flavoring agents, or one or more sweetening agents, such as sucrose or saccharin.

Oily suspensions may be formulated by suspending the active ingredients in an omega-3 fatty acid, a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents, such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient(s) (e.g., a compound of at least one of Formula (I) and/or an isomeric or tautomeric form of Formulas (II)-(X), respectively) in admixture with a dispersing or wetting agent, a suspending agent and one or more preservatives. Suitable dispersing or wetting agents and suspending agents are exemplified by those already mentioned above. Additional excipients, for example sweetening, flavoring and coloring agents, may also be present.

Syrups and elixirs containing the novel combination may be formulated with sweetening agents, for example glycerol, sorbitol or sucrose. Such formulations may also contain a demulcent, a preservative and flavoring and coloring agents.

Rectal

A suppository for rectal administration of the drug can be prepared by mixing the drug with a suitable non-irritating excipient such as cocoa butter, synthetic mono-, di- or triglycerides, fatty acids and polyethylene glycol (PEG) that is solid at ordinary temperatures (e.g., room temperature from 15-25° C.) but liquid at the rectal temperature. Accordingly, once rectally administered, the suppository excipient will melt in the rectum and release the subject eplerenone compound or its derivative(s).

### LIST OF REFERENCES

Lijnen, H. R.: Plasmin and matrix metalloproteinases in vascular remodeling, Thromb. Haemost. 86:324-333 (2001)

Shah, P. K., Galis, Z. S.: Matrix metalloproteinase hypothesis of plaque rupture: players keep piling up but questions remain, Circulation 104:1878-1880 (2001)

George, S. J.: Therapeutic potential of matrix metalloproteinase inhibitors in atherosclerosis, Expert Opin. Investig. Drugs, 9(5):993-1007 (2000)

Creemers, E. E., Cleutjens, J. P., Smits, J. F., Daemen, M. J.: Matrix metalloproteinase inhibition after myocardial infarction: a new approach to prevent heart failure? Circ. Res. 89:201-210 (2001)

Terrence M. Doherty, et al., Therapeutic Developments in Matrix Metalloproteinase Inhibition, review article, Expert Opin. Cir. Patents, 12(5) pp. 665-707, Ashley Publications (2002)

Li, Y. Y., Feng, Y., McTiernan C. F., et al., Downregulation of matrix metalloproteinase and reduction in collagen damage in the failing human heart after support with left-ventricular assist devices, Circulation, 104:1147-1152 (2001)

Mann D. L., Taegtmeyer H., Dynamic regulation of the extracellular matrix after mechanical unloading of the failing human heart: recovering the missing link in left ventricular remodeling, Circulation, 104:1089-1091 (2001)

Lee, R. T., Matrix metalloproteinase inhibition and the prevention of heart failure, Trends Cardiovasc. Med., 11:202-205 (2001)

Kim, H. E., Dalal, S. S., Young, E., Legato, M., Weisfeldt, M. L., D'Armento J., Disruption of the myocardial extracellular matrix leads to cardiac dysfunction, J. Clin. Invest., 108:857-866 (2000)

Spinale, F. G., Coker, M. L., Bond, B. R., Zellner, J. L., Myocardial matrix degradation and metalloproteinase activation in the failing heart: a potential therapeutic target, Cardiovasc. Res., 46:225-238 (2000)

Etoh, T., Joffs, C., Deschamps, A. M. et al., Myocardial and interstitial matrix metalloproteinase activity after acute myocardial infarction in pigs, Am J. Physiol. Heart Circ. Physiol. 281:H987-H994 (2001)

Stary, H. C., The sequence of cell and matrix changes in atherosclerotic lesion of coronary arteries in the first forty years of life, Eur. Heart J. 11(Suppl. E) 3:19 (1990)

Ducharme, A., Frantz, S., Aikawa, M., et al., Targeted deletion of matrix metalloproteinase-9 attenuates left ventricular enlargement and collagen accumulation after experimental myocardial infarction, J. Clin Invest. 106:55-62 (2000)

WO 01/05756 (2001)

WO 01/38301 (2001)

WO 01/05389 (2001)

WO 01/12611 (2001) and

WO 01/85680 (2001).

### EXAMPLES

The following examples describe embodiments of the invention. Other embodiments within the scope of the embodiments herein will be apparent to one skilled in the art from consideration of the specification or practice of the invention as disclosed herein. It is intended that the specification, together with the examples, be considered to be exemplary only, with the scope and spirit of the invention being indicated by the embodiments and the examples.

Zymography

Zymography was performed as described previously in Chadwick, V., Thomas, B. S., Mytsi, L., Coker, B. A., Zellner, J. L, Handy, J. R., et al. Increased Matrix Metalloproteinase Activity and Selective Upregulation in LV Myocardium From Patients with End-Stage Dilated Cardiomyopathy. Circulation 1998: 97; 1708-1715. Three groups of dogs were tested, each group containing 7 dogs (n=7).

Materials and Methods

Animals

Twenty-one healthy male and female mongrel dogs weighting 21-28 kg were obtained from Hodgkin's Kennels (Howell, Mich.). At the time of entry into the study, the age of the dogs ranged from 1-4 years. All animals were housed in a room with an ambient temperature of 22±1° C. on a 12 hours light/12 hours dark cycle in an animal care facility at Henry Ford Hospital, Detroit, Mich. Dogs received water to drink and Purina Proplan (K&S Pet Supplies, Westland, Mich.) dog chow at libitum. The present study was approved by the Henry Ford Hospital Care of Experimental Animals Committee and conformed to the “Position of the American Heart Association on Research Animals Use” and the Guiding Principles of the American Physiological Society.

Experimental Protocol

Chronic LV dysfunction was produced in 14 dogs (placebo dosed heart failure and eplerenone dosed heart failure canines) by multiple sequential intracoronary microembolizations using polystyrene latex microspheres (70-102 μM diameter, Polysciences, Inc., Warrington, Pa.) as previously described (Sabbah, H. N., Stanley, W. C., Sharov, V. G., Mishima, T., Tamimura, M., Benedict, et al. Effects of dopamine β-hydroxylase inhibition with nepicastat on the progression of left ventricular dysfunction and remodeling in dogs with chronic heart failure. Circulation 102 (2000): 1990-1995) to achieve a target LV ejection fraction of 30% to 40%. Coronary microembolizations were performed during sequential cardiac catheterizations under general anesthesia and sterile conditions. A combination of oxymorphone (0.22 mg/kg, i.v., Amerisource, Toledo, Ohio), diazepam (0.17 mg/kg, iv., Amerisource, Toledo, Ohio), and pentobarbital sodium (150-250 mg, i.v., Amerisource, Toledo, Ohio) was used to achieve a surgical plane of anesthesia. This anesthetic regimen has been shown to be effective in preventing the tachycardia, systemic hypertension, and myocardial depression associated wit the use of pentobarbital alone. (Sabbah, H. N., Shimoyama, H., Kono, T., Gupta, R. C., Sharov, V. G., Scicli, et al. Effects of long-term monotherapy with enalapril, metropolol, and digoxin on the progression of left ventricular dysfunction and dilation in dogs with reduced ejection fraction. Circulation 89 (1994):2852-2859.) In the above-referenced 14 dogs, coronary microembolizations were discontinued when LV ejection fraction reached 30-40% as determined angipgraphically. To achieve this target ejection fraction, dogs underwent an average of 5.9 microembolization procedures performed over an average period of 6.6 weeks. A minimum of 1 wk was allowed between embolizations. In all instances, selective microembolization of the left coronary artery was achieved by injections of microspheres into the left anterior descending coronary artery and circumflex coronary artery. The right coronary artery was not embolized as it only perfuses <70% of the right ventricular free wall and none of the LV myocardium. Two weeks after the last coronary microembolization, when infarct healing was complete, all dogs underwent a left and right heart catheterization. After 24 hours, the above-referenced 14 dogs were randomized into 2 groups and treated for 3 months. One group (n=7) received eplerenone (eplerenone dosed heart failure canines) 20 mg/kg/day, (dosed at 10 mg/kg bid, po) and a second group (n=7) received no treatment and served as control (placebo dosed heart failure canines). No other agents were administered during the study.

Zymographic Evaluation

On the day of sacrifice, all 21 animals were weighted and the chest was opened through a left thoracotomy, pericardium was opened and the heart rapidly removed and placed in ice-cold, Tris buffer (pH 7.4). The left and right ventricles were separated. A 2-mm thick transverse slice was obtained from the LV, snap frozen in liquid nitrogen and stored at −70° C. until use. Tissue sections were powdered and homogenization buffer (1% Triton, 25 mM HEPES, 0.15 M NaCl, 2 mM EDTA) was added to a final concentration of 300 mg tissue/mL, and agitated with a Polytron homogenizer. Homogenates were centrifuged at 6000 rpm, 4° C. for 20 minutes and supernatants were used for zymographic analysis. Protein concentrations of the homogenates were determined using BCA protein assay (Pierce, Rockford, Ill.) using albumin as a standard according to manufacturer's instructions.

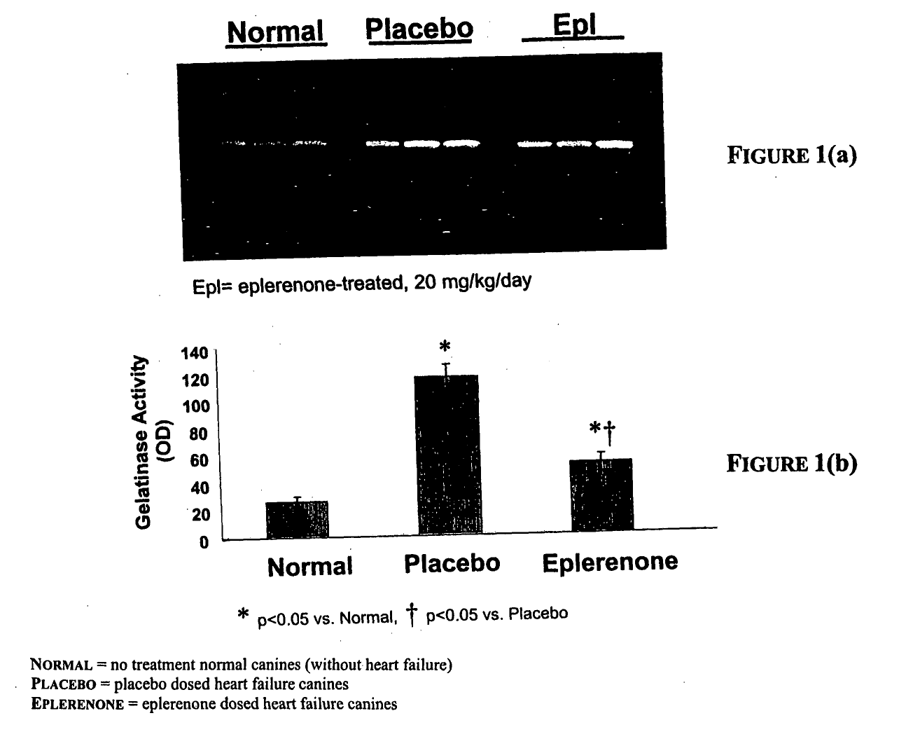

Thereafter, 50 μg aliquots of heart homogenate were diluted with 2×SDS sample buffer and loaded onto 10% zymogram gels containing 0.1% gelatin substrate (Invitrogen, Carlsbad, Calif.). Electrophoresed gels were washed in 2.5% (v/v) Triton X-100 and incubated overnight in 1× developing buffer (Invitrogen). Gels were stained using 0.5% Coomassie Brilliant Blue R250 (w/v) and destained until zones of proteolysis were clearly delineated. Gelatinase activity was quantitated using densitometry and activity is represented as optical density. See FIGS. 1a and 1b.

### REFERENCES

All references cited in this specification, including without limitation, all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinency of the cited references.

What is claimed is:

1. A method for preventing an increase in matrix metalloproteinase (MMP) activity or reducing MMP activity in a subject, said method comprising the step of:
a) administering to said subject a therapeutically effective amount of an aldosterone blocker.

2. The method of claim 1, wherein said aldosterone antagonist comprises eplerenone.

3. The method of claim 1, wherein said aldosterone antagonist comprises spironolactone.

4. The method of claim 1, wherein said MMP activity is modulated in myocardial tissue.

5. The method of claim 1, wherein said MMP activity is modulated in left ventricular tissue.

6. The method of claim 1, wherein said MMP activity is modulated in the tissue of a member selected from the group consisting of heart, kidney and brain.

7. The method of claim 1, wherein said MMP activity is modulated in a coronary artery.

8. The method of claim 1, wherein said MMP activity is MMP-2 activity.

9. The method of claim 1, wherein said MMP activity is MMP-9 activity.

10. The method of claim 1, wherein said MMP activity is MMP-13 activity.

11. The method of claim 1, wherein said MMP activity is MMP-2 activity, MMP-9 activity or MMP-13 activity.

12. The method of claim 1, wherein said MMP activity is modulated in said subject suffering from hypertension.

13. The method of claim 1, wherein said MMP activity is modulated in said subject suffering from heart failure.

14. The method of claim 13, wherein said heart failure is selected from the group consisting of class-II, class-III and class-IV heart failure.

15. The method of claim 1, wherein said MMP activity is modulated in said subject suffering from cardiac fibrosis.

16. The method of claim 1, wherein said MMP activity is modulated in said subject suffering from atherosclerosis.

17. The method of claim 1, wherein said MMP activity is modulated in said subject suffering from enlargement of the heart.

18. The method of claim 1, wherein said MMP activity is modulated in said subject suffering from left ventricular dilation.

19. The method of claim 1, wherein said MMP activity is modulated in said subject suffering from progressive left ventricular failure.

20. The method of claim 19, wherein said MMP activity is modulated in said subject having a left ventricular ejection fraction less than about 40%.

21. The method of claim 1, wherein said subject is a mammal.

22. The method of claim 21, wherein said mammal is a human.

23. The method of claim 22, wherein said human has symptoms of or has had symptoms of a condition selected from the group consisting of heart failure, renal disease, stroke, diabetes and syndrome X.

24. The method of claim 2, wherein said administering step comprises administering a daily dose of said eplerenone from about 25 mg to about 400 mg.

25. The method of claim 24, wherein said daily dose is provided in a single daily dose.

26. The method of claim 24, wherein said daily dose is provided in multiple divided doses.

27. The method of claim 24, wherein said daily dose is administered orally.

28. The method of claim 1, wherein said aldosterone blocker inhibits said MMP activity.

29. The method of claim 2, wherein said eplerenone inhibits said MMP activity.

30. The method of claim 1, wherein said aldosterone blocker is an epoxy-steroidal aldosterone blocker.

31. The method of claim 30, wherein said epoxy-steroidal aldosterone blocker is combined with a pharmaceutically acceptable carrier.

32. The method of claim 22, wherein said human has symptoms of or has had symptoms of syndrome X, atherosclerosis or myocardial infarction.

33. The method of claim 22, wherein said human has symptoms of or has had symptoms of coronary arterial disease.

34. The method of claim 1, wherein the aldosterone blocker is a selective aldosterone blocker.

35. The method of claim 1, wherein said MMP activity is modulated in renal tissue.

36. The method of claim 1, wherein said MMP activity is modulated in the vascular tissue of a member selected from the group consisting of heart, kidney and brain.

37. The method of claim 22, wherein said mammal has symptoms of or has had symptoms of a condition selected from the group consisting of heart failure, renal disease, stroke, diabetes and syndrome X.

38. The method of claim 32, wherein said mammal has symptoms of or has had symptoms of syndrome X, atherosclerosis or myocardial infarction.

39. The method of claim 33, wherein said mammal has symptoms of or has had symptoms of coronary arterial disease.
